(19) United States
(12) Patent Application Publication
(10) Pub. No.: US 2009/0191291 A9
(43) Pub. Date: Jul. 30, 2009

(54) Cosmetology product for skin and hair

(76) Inventor: Tina Ekker, Richfield, UT (US)

Correspondence Address: MORRISS OBRYANT COMPAGNI, P.C., 734 EAST 200 SOUTH, SALT LAKE CITY, UT, 84102, US

(21) Appl. No.: 11/641,223

(22) Filed: Dec. 19, 2006

Related U.S. Application Data
(60) Provisional application No. 60/751,714, filed on Dec. 19, 2005.

(51) Int. Cl. A61K 36/886; A61Q 5/00; A61P 25/00

(52) U.S. Cl. 424/744

(57) ABSTRACT

The invention provides compositions for external application to the skin and/or hair. The composition may be used to treat burns, particularly minor burns, sunburns, eczema, psoriasis, dry or bleeding cuticles, dry or rough skin, athletes foot, razor rash or razor burn, cold sores, dry or chapped lips, dry or frayed hair, and/or combinations thereof.

### TECHNICAL FIELD

This invention relates to topical compositions containing silicone emollients, such as dimethicone and/or cyclomethicone, and methods of use for regulating the condition of skin or hair, especially conditions related to hydration.

### BACKGROUND

As a result of several years of illness, where the inventor was diagnosed with eczema and contact dermatitis, my skin and hair were left in bad shape. In addition, many of the over-the-counter products that were used to treat these conditions produced an allergic reaction. Furthermore, the potential side affects caused by the prescription medications were undesirable, e.g., skin cancer with prolonged exposure to the sun. Therefore, through much trial and error, the present invention was created to overcome the limitations found in current over-the-counter medications and to treat eczema or dermatitis.

Cosmetology products for use on skin, e.g., a lotion, are known in the art. Cosmetology products for use on hair, e.g., shampoo, conditioner, hair gel and hair spray, are also well known in the art. Likewise, bath oils and salts are well known in the art. However, considerable room for improvement exists. In particular, present formulations are typically intended for application to either hair or skin, but not both.

Therefore, it would be desirable to have a cosmetology solution that could be used as a lotion for smoothing skin when applied directly, or as a hair detangler that improves shine when applied to hair in a wet or dry condition, or that could be used as a bath oil for indirectly smoothing skin. In addition, a cosmetology solution that could treat numerous disease conditions affecting the skin would be desirable.

Accordingly, the present invention provides an improved cosmetology solution for treatment of skin and/or hair.

### SUMMARY OF THE INVENTION

The invention provides compositions for external application to the skin and/or hair. A composition of the invention may be used to treat burns, particularly minor burns, sunburns, eczema, psoriasis, dry or bleeding cuticles, dry or rough skin, athletes foot, razor rash or razor burn, cold sores, dry or chapped lips, dry or frayed hair, and/or combinations thereof. A composition of the invention may also be used to treat pain associated with the skin or joints of the subject

Compositions of the invention include a topical composition comprising: a mixture of dimethicone and cyclomethicone, wherein the mixture is from about 60:40 to about 70:30 (vol.:vol.) dimethicone:cyclomethicone; mineral oil; Aloe vera gel; a vitamin E source; a vitamin A source; and water.

The mineral oil, Aloe vera gel, and/or vitamin E source may be in an amount from about 0.2% to about 4% of the volume of the dimeticone and cyclomethicone mixture. The vitamin A source may be in an amount from about 0.1% to about 2% of the volume of the dimeticone and cyclomethicone mixture.

Optionally, the compositions of the invention may contain a scented oil, for example, in an amount of from about 0.1% to about 2% of the volume of the dimeticone and cyclomethicone mixture.

The invention also relates to a method of maintaining or improving a skin or hair condition by applying a composition of the invention to the skin and/or hari of a subject, for example, a mammal, such as a human, thought to be in need of such treatment.

### BRIEF DESCRIPTION OF THE DRAWINGS

FIG. 1 shows the affect of three days of treatment on eczema, with the photograph on Nov. 30, 2006, illustrating the skin prior to treatment and the photographs on Dec. 2 and 3 illustrate the beneficial effect of the composition.

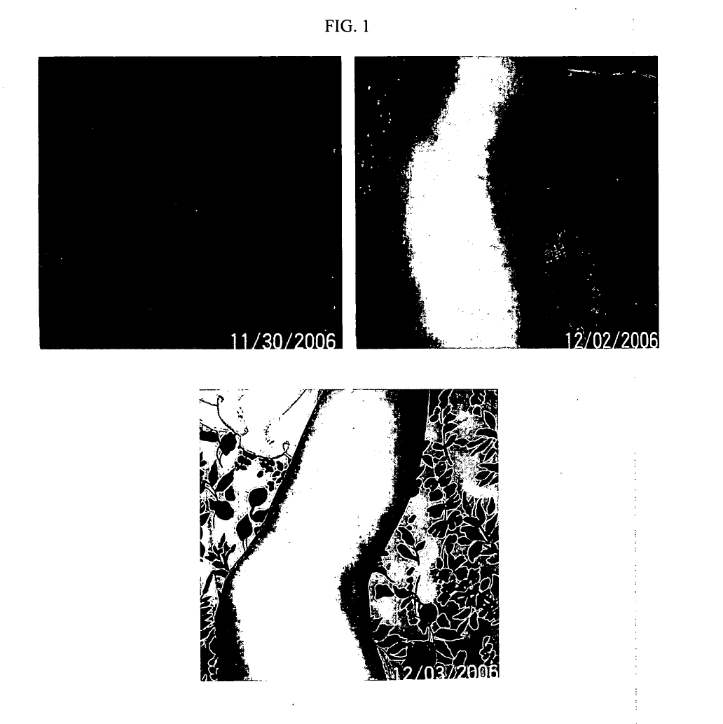

### DETAILED DESCRIPTION OF THE INVENTION

The invention relates to a topical composition that is beneficial in the treatment of eczema, psoriasis, and dermatitis. Surprisingly, the composition is also effective in the treatment of hair and other skin conditions.

Use of the compositions has been found to make the user's hair more manageable, easier to comb out (detangled), softer, in better condition, improve split ends, and improve the shine or appearance of the hair. The formulation also works extremely well for hair that is blow dried, hot flat ironed and curled with hot rollers, particularly, when the product is applied prior to the use of these items.

Use of the product on the skin showed that it was able to relieve dry skin, make the skin softer to the touch, and reduce the signs of aging by reducing the appearance of fine lines and wrinkles on the chest, face and neck. The product is also beneficial after shaving to help eliminate chaffing and/or razor rash keeping the skin soft and smooth. In addition, the product provides a bath treatment that may be added to warm bath water to soften and smooth the skin, and may remove the need to use a cream or a lotion after bathing.

In particular, the invention relates to a leave-in conditioner for hair, an oil, cream or lotion for the skin, and a bath oil. The oil, cream or lotion for the skin may be used alone or under make-up and other moisturizers or creams as desired.

In addition, the compositions of the invention are believed to be effective for the treatment for eczema and dermatitis.

As used herein and in the appended claims, the singular forms, for example, “a”, “an”, and “the,” include the plural, unless the context clearly dictates otherwise. For example, reference to “a mineral oil” includes a plurality of such oils, and reference to a “vitamin E source” includes a reference to a plurality of similar vitamin E sources, and equivalents thereof.

As used herein and in the appended claims, “about” means reasonably close to, or approximately, a little more or less than the stated number or amount.

As used herein and in the appended claims, “Aloe Vera,” means a compound or composition comprising an extract, exudate, gel, juice, sap or latex derived from a plant of the Aloe genus (e.g., “Aloe vera,” with Aloe barbadensis Miller and Aloe aborescens being the most widely used species for commercial purposes), including simple leaf pressings or cuttings, preferably, Aloe vera is a clear colorless semi-solid Aloe Gel that has been stabilized. Aloe Vera is particularly rich in vitamins A, C, & E.

As used herein, “dimethicone,” means a mixture of fully methylated, linear siloxane polymers conforming to the general chemical structure: R₃—Si—O—[SiOR₂]ₙ—Si—R₃, wherein R is an alkyl group having from one to thirty carbon atoms, preferably R is methyl or ethyl, and wherein n is an integer from 1 to about 10,000 depending on the desired molecular weight. In an exemplary embodiment, the dimethicone is of medium viscosity, e.g., 50 centistokes (cSt) to 1,000 cSt, including 50, 100, 200, 350, 500, or 1,000 cSt dimethicone. Dimethicone for use in the composition may be obtained from Clearco Products, particularly 350 cSt. As will be apparent in light of the teachings herein dimethicone having a low viscosity, medium viscosity, high viscosity and mixtures thereof may also be utilized (see also U.S. Pat. No. 6,238,678).

As used herein, “cyclomethicone,” means a mixture of unmodified silicones that generally possess a cyclical structure. Cyclomethicone may be obtained from Clearco Products, particularly a volatile polydimethylcyclosiloxane (cyclomethicone) sold by Clearco Products as Cyclo-2244 (D-4), which is composed mainly of octamethylcyclotetrasiloxane (CAS#556-67-2). As will be apparent in light of the teachings herein D-4, D-5, D-6 and mixtures thereof may also be utilized (see, also U.S. Pat. No. 6,238,678).

As used herein, “mineral oil” means an oil or liquid petrolatum that is chemically inert, transparent, colorless oil composed mainly of alkanes and cyclic paraffins.

As used herein, a “vitamin E source” means an oil derived from a source having vitamin E or an oil to which vitamin E has been added, wherein the oil is enriched for vitamin E. For example, vitamin E sources include, but are not limited to, Soybean oil, Coconut oil, Wheat germ oil, Sunflower oil, Hazelnut oil, Walnut oil, Peanut oil, and Olive oil. In an exemplary embodiment, a “vitamin E source” is a “liquid vitamin E” based on a soybean oil having approximately 12,000 international units (IUs) per two ounces, or 12,000IUs per 8 oz, where one IU is equal to 0.67 milligram of d-alpha-tocopherol.

As used herein, a “vitamin A source” means an oil derived from a source having vitamin A or an oil to which vitamin A has been added, wherein the oil is enriched for vitamin A. For example, vitamin A sources include, but are not limited to, a liquid vitamin A utilizing Soybean oil as the carrier or vehicle and having vitamin A derived from fish liver in the form of retinol palmitate.

As used herein, a “scented oil” means an aromatic oil or an essential oil. As used herein, an “essential oil” means any volatile oil that imparts the characteristic odor of a plant. Examples of essential oils includes, but is not limited to, Angelica Root, Anise, Peru Balsam, Basil, Bay, Bay Laurel, Beeswax, Benzoin, Bergamot, Bergamot Mint, Bois-de-rose, Boronia, Cajeput, Cardamum, Carrot Seed, Cedarwood, Chamomile, Cinnamon, Citronella, Clary Sage, Clove, Coriander, Cypress, Dill, Elemi, Eucalyptus, Fennel, Fir Needle, Frankincense, Galbanum, Geranium, Ginger, Grapefruit, Helichrysum, Hyssop, Immortelle, Jasmine, Juniper Berry, Kanuka, Lavender, Lavendin, Lemon, Lemongrass, Lime, Linden Blossom, Mandarin, Manuka, Marjoram, May Chang, Myrrh, Myrtle, Neroli, Niaouli, Nutmeg, Oakmoss, Olibanum, Orange, Oregano, Palmarosa, Parsley, Patchouli, Pepper, Peppermint, Petitgrain, Pine, Ravensara, Rose, Rosemary, Rosewood, Sandalwood, Spearmint, Spikenard, Spruce, Tagetes, Tangerine, Tea Tree, Thyme, Tobacco, Tuberose, Vanilla, Vetiver, Violet Leaf, Yarrow, and/or Ylang Ylang.

As used herein, a “Zinc source” means a source of the element zinc (Zn), including, but not limited to, zinc derived from dairy products, beans and lentils, yeast, nuts, seeds and wholegrain cereals, where pumpkin seeds provide one of the most concentrated natural sources of food based zinc. Optionally, the zinc source is dissolved or suspended in a liquid, oil or water, prior to being added to the composition of the invention.

As used herein and in the appended claims, “Treating” or “Treatment” does not mean a complete cure. It means that the symptoms of the underlying disease are reduced, delayed, or prevented, or that one or more of the underlying cellular, physiological, or biochemical causes or mechanisms causing the symptoms are reduced, delayed, or prevented. It is understood that reduced, delayed, or prevented, as used in this context, means relative to the untreated state of the disease, including the molecular state of the disease, not just the physiological state of the disease.

As used throughout the specification, for the sake of brevity, “comprising,” “including,” “containing,” “characterized by,” and grammatical equivalents thereof are inclusive or open-ended terms that do not exclude additional, unrecited elements or method steps, but should also be understood to include the more restrictive or closed terms “consisting of” and “consisting essentially of.” For example, a sentence stating that “the invention comprises . . . ” is to be understood as including the alternative statements that “the invention consists of . . . ” and “the invention consists essentially of . . . ” and grammatical equivalents thereof.

Ranges can be expressed herein as from “about” one particular value to “about” another particular value. When values are expressed as approximations, by use of the antecedent “about,” it will be understood that the particular value forms another embodiment. It will be further understood that the endpoints of each of the ranges are significant both in relation to the other endpoint, and independently of the other endpoint. It is also to be understood that a range expressed as from about one particular value to about another particular value includes all values and ranges therein.

In an exemplary embodiment, lavender oil is used to prepare a composition useful for treating pain, particularly pain associated with arthritis.

Exemplary ratios of the dimethicone to the cyclomethicone are from about 60:40 to about 70:30 (vol.:vol.), or about 61:39 to about 69:31 (vol.:vol:.), or about 62:38 (vol.:vol.) to about 68:32, or about 63:37 to about 67:33, or about 60:40, about 61:39, about 62:38, about 63:37, about 64:36, about 65:35, about 66:34, about 67:33 (3:1), about 68:32, about 69:31, or about 70:30.

In addition to the dimethicone and cyclomethicone, the content of other ingredients in this mixture may be from about 0.2% to about 15% (vol./vol.) of the mixture, or from about 1% to about 14% (vol./vol.), or from about 3% to about 13% (vol./vol.), or about 4% to about 13%, or about 6% to about 13%, or about 12% to about 13%, or about 1%, about 3%, about 4%, about 6%, about 12%, about 13%, or about. 14% of the volume of the dimeticone and cyclomethicone mixture (e.g., 12 ounces of dimethicone and 6 ounces of cyclomethicone yields approximately 480 ml of the dimethicone and cyclomethicone mixture (assuming 30 ml per fluid ounce) and adding 7.50 mls each of mineral oil, Aloe vera gel, and vitamin E oil adds about 4.7% (≈1.6% each) relative to the volume of the dimethicone/cyclomethicone mixture).

Other ingredients include, Mineral Oil, Aloe Vera Gel, a Vitamin E source (for example, an oil containing soybean oil, tocopheryl acetate, and coconut oil), a Vitamin A source (for example, an oil enriched for vitamin A (e.g., retinol palmitate) containing soybean oil and fish liver oil), and water (for example, spring or mineral water).

In another exemplary embodiment, mineral oil is added at a concentration of between about 0.2% to about 4% (Vol.:Vol.), or about 0.3% and about 3.2% (Vol.:Vol.), or about 0.3% to about 4%, or about 3% to about 4%, or about 1% to about 2% (Vol.:Vol.), or about 0.3% (Vol.:Vol.), or about 1.6% (Vol.:Vol.), or about 3.1% (Vol.:Vol.), with Vol.:Vol. being the volume of the mineral oil relative to the volume of the dimethicone/cyclomethicone mixture (e.g., adding 3.5 ml of mineral oil to 8 oz of the dimethicone/cyclomethicone mixture equals adding about 1.5% mineral oil).

In another exemplary embodiment, Aloe vera is added at a concentration of between about 0.2% to about 4% (Vol.:Vol.), or about 0.3% and about 3.2% (Vol.:Vol.), or about 0.3% to about 4%, or about 3% to about 4%, or about 1% to about 2% (Vol.:Vol.), or about 0.3% (Vol.:Vol.), or about 1.6% (Vol.:Vol.), or about 3.1% (Vol.:Vol.), with Vol.:Vol. being the volume of the Aloe vera relative to the volume of the dimethicone/cyclomethicone mixture.

In another exemplary embodiment, a vitamin E source is added at a concentration of between about 0.2% to about 4% (Vol.:Vol.), or about 0.3% and about 3.2% (Vol.:Vol.), or about 0.3% to about 4%, or about 3% to about 4%, or about 1% to about 2% (Vol.:Vol.), or about 0.3% (Vol.:Vol.), or about 1.6% (Vol.:Vol.), or about 3.1% (Vol.:Vol.), with Vol.:Vol. being the volume of the vitamin E source relative to the volume of the dimethicone/cyclomethicone mixture.

In another exemplary embodiment, a vitamin A source is added at a concentration of between about 0.1% to about 2% (Vol.:Vol.), or about 1% to about 2%, or about 0.3% to about 1.6%, or about 0.2% and about 1.6% (Vol.:Vol.), or about 0.26% (Vol.:Vol.), or about 0.78% (Vol.:Vol.), or about 1.56% (Vol.:Vol.), with Vol.:Vol. being the volume of the vitamin A source relative to the volume of the dimethicone/cyclomethicone mixture.

In another exemplary embodiment, a scented oil is added at a concentration of between about 0.1% to about 2% (Vol.:Vol.), or about 1% to about 2%, or about 0.2% and about 1.6% (Vol.:Vol.), or about 0.26% (Vol.:Vol.), or about 0;78% (Vol.:Vol.), or about 1.56% (Vol.:Vol.), with Vol.:Vol. being the volume of the scented oil relative to the volume of the dimethicone/cyclomethicone mixture.

In another exemplary embodiment, water is added at a concentration of between about 0.05% to about 0.5% (Vol.:Vol.), or about 0.1% to about 0.3%, or about 0.2% to about 0.3%, or about 0.13% to about 0.26%, or about 0.2%, or about 0.13%, or about 0.26% (Vol.:Vol.), with Vol.:Vol. being the volume of the water relative to the volume of the dimethicone/cyclomethicone mixture.

In another exemplary embodiment, an additive selected from the group consisting of zinc, iodine, rice extract, kelp extract, pumpkin seed extract and mixtures thereof may be added to any of the compositions of the invention. In another exemplary embodiment, a source of zinc is dissolved or suspended in a carrier and the dissolved or suspended solution is added to the composition.

In another exemplary embodiment, an essential oil is added as desired for scent or softening properties to a composition of the invention comprising dimethicone, cyclomethicone, mineral oil, aloe vera gel, a vitamin E source, and a vitamin A source. Optionally, a zinc source may be added.

In another exemplary embodiment, the composition comprises dimethicone and cyclomethicone in a ratio of about 3:1 or about 5:3 with one or more additional agents added according to the concentrations illustrated in Table 1:

TABLE 1

| | Milliliters per 8 ounces (≈240 ml) | Percentage relative to |
|---|---|---|
| | of dimethicone and | dimethicone/cyclomethicone |
| Agent | cyclomethicone¹ | mixture² |
| Mineral oil | 7.5, 3.5, 3.25, 2.5, 1.5, or | 3.1%, 1.6%, 1.4%, 1.0%, 0.6%, or |
| | 0.75 ml | 0.3% |
| Aloe Vera Gel | 7.5, 3.5, 3.25, 2.5, 1.5, or | 3.1%, 1.6%, 1.4%, 1.0%, 0.6%, or |
| | 0.75 ml | 0.3% |
| Vitamin E oil (e.g., | 7.5, 3.5, 3.25, 2.5, 1.5, or | 3.1%, 1.6%, 1.4%, 1.0%, 0.6%, or |
| 6,000 IU per oz) | 0.75 ml | 0.3% |
| Vitamin A oil | 3.75, 1.9, 1.25, 1.0, or 0.6 ml | 1.6%, 0.8%, 0.5%, 0.4%, or 0.3% |
| Scented oil | 0.3 ml to about 3.25 | 0.1% to about 1.4% |
| A zinc solution | 3.75 ml | 1.6% |
| Water | About 0.6 to about 0.3 ml | 0.3% to 0.1% |

¹All values are approximate.
²All values are approximate and rounded to the nearest tenth of a percent.

In another exemplary embodiment, the composition comprises dimethicone and cyclomethicone in a ratio of about 1:3 or about 3:5, which is preferably utilized as a hand lotion or for the treatment of cuticles, wherein the composition may have one or more additional agents added according to the concentrations illustrated in Table 2:

TABLE 2

| | Milliliters per 8 ounces (≈240 ml) | Percentage relative to |
|---|---|---|
| | of dimethicone and | dimethicone/cyclomethicone |
| Agent | cyclomethicone¹ | mixture² |
| Mineral oil | 7.5, 3.5, 3.25, 2.5, 1.5, or | 3.1%, 1.6%, 1.4%, 1.0%, 0.6%, or |
| | 0.75 ml | 0.3% |
| Aloe Vera Gel | 7.5, 3.5, 3.25, 2.5, 1.5, or | 3.1%, 1.6%, 1.4%, 1.0%, 0.6%, or |
| | 0.75 ml | 0.3% |
| Vitamin E oil (e.g., | 7.5, 3.5, 3.25, 2.5, 1.5, or | 3.1%, 1.6%, 1.4%, 1.0%, 0.6%, or |
| 6,000 IU per oz) | 0.75 ml | 0.3% |
| Vitamin A oil | 3.75, 1.9, 1.25, 1.0, or 0.6 ml | 1.6%, 0.8%, 0.5%, 0.4%, or 0.3% |
| Scented oil | 0.3 ml to about 3.25 | 0.1% to about 1.4% |
| A zinc solution | 3.75 ml | 1.6% |
| Water | About 0.6 to about 0.3 ml | 0.3% to 0.1% |

¹All values are approximate.
²All values are approximate and rounded to the nearest tenth of a percent.

In another exemplary embodiment, the composition comprises a mixture of dimethicone and cyclomethicone, Mineral Oil, Aloe Vera Gel, a Vitamin E source, a Vitamin A source, and water. In another exemplary embodiment, the composition comprises a mixture of dimethicone and cyclomethicone, Mineral Oil, Aloe Vera Gel, a Vitamin E source, a Vitamin A source, a scented oil and water. In another exemplary embodiment, the composition comprises a mixture of dimethicone and cyclomethicone, Mineral Oil, Aloe Vera Gel, a Vitamin E source, a Vitamin A source, a Zinc source and water. In another exemplary embodiment, the composition comprises a mixture of dimethicone and cyclomethicone, Mineral Oil, Aloe Vera Gel, a Vitamin E source, a Vitamin A source, a Zinc source, a scented oil and water.

In another exemplary embodiment, the composition comprises a mixture of dimethicone and cyclomethicone, Mineral Oil, Aloe Vera Gel, a Vitamin E source, a Vitamin A source, water and an essential oil derived from a plant selected from the group consisting of Angelica Root, Anise, Peru Balsam, Basil, Bay, Bay Laurel, Beeswax, Benzoin, Bergamot, Bergamot Mint, Bois-de-rose, Boronia, Cajeput, Cardamum, Carrot Seed, Cedarwood, Chamomile, Cinnamon, Citronella, Clary Sage, Clove, Coriander, Cypress, Dill, Elemi, Eucalyptus, Fennel, Fir Needle, Frankincense, Galbanum, Geranium, Ginger, Grapefruit, Helichrysum, Hyssop, Immortelle, Jasmine, Juniper Berry, Kanuka, Lavender, Lavendin, Lemon, Lemongrass, Lime, Linden Blossom, Mandarin, Manuka, Marjoram, May Chang, Myrrh, Myrtle, Neroli, Niaouli, Nutmeg, Oakmoss, Olibanum, Orange, Oregano, Palmarosa, Parsley, Patchouli, Pepper, Peppermint, Petitgrain, Pine, Ravensara, Rose, Rosemary, Rosewood, Sandalwood, Spearmint, Spikenard, Spruce, Tagetes, Tangerine, Tea Tree, Thyme, Tobacco, Tuberose, Vanilla, Vetiver, Violet Leaf, Yarrow, Ylang Ylang and combinations thereof. Optionally, a Zinc source may be added to this embodiment.

In another exemplary embodiment, the composition comprises a mixture of dimethicone and cyclomethicone, Mineral Oil, Aloe Vera Gel, a Vitamin E source, a Vitamin A source, water and an essential oil derived from lavender, wherein the composition may be used to treat pain.

Compositions according to the invention may be made by mixing at moderate speed thereby introducing air into the composition. In an exemplary embodiment, the compositions according to the invention are mixed at moderate speed so as to introduce air into the composition wherein approximately 16 oz of starting material yields a volume of approximately 16.1 to about 16.8 oz, or about 16.1, 16.2, 16.3, 16.4, 16.5, 16.6, 16.7, or 16.8 oz.

The invention also relates to a method of reducing symptoms in a subject believed to be suffering from or susceptible to a symptomatic state susceptible to treatment by a composition according to the invention, the method comprising: administering the composition according to the invention to an area of the skin or hair though to be in need of such treatment.

The composition of the invention may be used to treat burns, particularly minor burns, such as first or second degree burns, sunburns, eczema, psoriasis, dry or bleeding cuticles, dry or rough skin, athletes foot, razor rash or razor burn, cold sores, dry or chapped lips, and/or dry or frayed hair.

The compositions of the invention will now be illustrated by way of the following illustrative Examples.

### EXAMPLE I

An exemplary embodiment of the invention includes the following formulation:
6 ounces of Dimethicone;
2 ounces of Cyclomethicone;
7.50 ml Mineral Oil;
7.50 ml Aloe Vera Gel;
7.50 ml of a Vitamin E source, such as liquid vitamin E);
3.75 ml Vitamin A oil;
3.75 ml of a Zinc source; and
0.625 ml water.

### EXAMPLE II

Another exemplary embodiment of the invention includes the following formulation:
12 ounces of Dimethicone;
4 ounces of Cyclomethicone;
7.50 ml Mineral Oil;
7.50 ml Aloe Vera Gel (e.g., having vitamin A, C, & E)
7.50 ml Vitamin E oil;
3.75 ml Vitamin A oil;
0.625 ml of perfume scented oil, (e.g., vanilla, musk, lilac, fruit scent, etc.); and
0.625 ml water.

### EXAMPLE III

Another exemplary embodiment of the invention includes the following formulation:
12 ounces of Dimethicone;
4 ounces of Cyclomethicone;
1.50 ml Mineral Oil;
1.50 ml Aloe Vera Gel;
1.50 ml of a Vitamin E source (e.g., a soybean based vitamin E oil);
1.25 ml of a Vitamin A source (e.g., a soybean based vitamin A oil where the vitamin A is derived from fish liver oil); and
0.625 ml water.

### EXAMPLE IV

Another exemplary embodiment of the invention includes the following formulation:
12 ounces of Dimethicone;
4 ounces of Cyclomethicone;
1.50 ml Mineral Oil;
1.50 ml Aloe Vera Gel;
1.50 ml of a Vitamin E source;
1.25 ml of a Vitamin A source; and
0.625 ml water.

### EXAMPLE V

The compositions of the invention may be formulated as described in the present Example. However, as will be recognized by a person of skill in the art, the present method may be scaled up using equipment and techniques known in the art.

The compositions of the invention are formulated by adding the silicone (dimethicone and cyclomethicone) ingredients as they are listed in Examples VI to X, mixing and or blending in a suitable container, blending at a high speed for not less than one minute, adding additional ingredients as listed in Examples VI to X one at a time and mixing and or blending in the same suitable container for not less than one minute after each ingredient is added, when all ingredients are added, blend or mix all ingredients together in same suitable container for not less than one minute, and transferring the composition into individual suitable bottles or containers and sealing the bottle or container.

### EXAMPLE VI

Another exemplary embodiment of the invention includes the following formulation:
6 ounces of 350 cSt Dimethicone;
2 ounces of D-4 Cyclomethicone;
7.50 ml Mineral Oil;
7.50 ml Aloe Vera Gel;
7.50 ml Vitamin E oil;
3.75 ml Vitamin A oil;
3.25 ml lavender essential oil; and
0.625 ml spring water.

This embodiment is preferably used as a topical agent on a subject's skin.

### EXAMPLE VII

Another exemplary embodiment of the invention includes the following formulation:
6 ounces of 350 cSt Dimethicone;
2 ounces of D-4 Cyclomethicone;
7.50 ml Mineral Oil;
7.50 ml Aloe Vera Gel;
7.50 ml Vitamin E oil;
3.75 ml Vitamin A oil;
3.25 ml lavender essential oil;
3.25 ml of a zinc source; and
0.625 ml spring water.

This embodiment is preferably used as a topical agent on a subject's skin.

### EXAMPLE VIII

Another exemplary embodiment of the invention includes the following formulation:
5 ounces of 350 cSt Dimethicone;
3 ounces of D-4 Cyclomethicone;
2.50 ml Mineral Oil;
2.50 ml Aloe Vera Gel;
2.50 ml Vitamin E oil;
1.25 ml Vitamin A oil; and
0.625 ml spring water.

This embodiment is preferably used to detangle, manage split ends and/or condition a subject's hair.

### EXAMPLE IX

Another exemplary embodiment of the invention includes the following formulation:
6 ounces of 350 cSt Dimethicone;
2 ounces of D-4 Cyclomethicone;
3.50 ml Mineral Oil;
3.50 ml Aloe Vera Gel;
3.50 ml Vitamin E oil;
1.25 ml Vitamin A oil; and
0.625 ml spring water.

This embodiment is preferably used as a bath oil.

### EXAMPLE X

Another exemplary embodiment of the invention includes the following formulation:
3 ounces of 350 cSt Dimethicone;
5 ounces of D-4 Cyclomethicone;
1.50 ml Mineral Oil;
1.50 ml Aloe Vera Gel;
1.50 ml Vitamin E oil;
1.00 ml Vitamin A oil; and
0.625 ml spring water.

This embodiment is preferably used as a hand lotion, particularly for the treatment of dry or over grown cuticles.

### EXAMPLE XI

Another exemplary embodiment of the invention includes the use of the composition to treat psoriasis and/or eczema. The composition was applied to individuals suffering from psoriasis and/or eczema, including individuals having extreme cases of psoriasis and/or eczema resulting in itchy, scaly, rough, dry, sore, patchy plaques, bleeding, and/or oozing areas on the skin. After several treatments individuals reported that the skin appeared to be healing, that the soreness was relieved, and that the itching was absent. Most individuals who continued use of the composition for a period of four to six weeks reported that no psoriasis or eczema was apparent and the absence of further outbreaks.

The product was topically applied to an elderly lady believed to be suffering from psoriasis. This individual reportedly had been undergoing medical treatment for this condition for a period of four (4) years prior to the present treatment with little relief. The prescribed treatment was said to require topical treatment at night, which comprised wrapping her arms and applying hot packs to them to prevent bleeding as she slept. Two weeks after applying the present invention her face was clear, and within 4 weeks significant improvements in her arms were reported.

### EXAMPLE XII

Another exemplary embodiment of the invention includes the use of the composition to treat minor burns. The composition was applied to minor burns on individuals, particularly those causing small red marks or blisters on the skin, who reported that the burn sensation immediately went away and that with continued use the burn healed with little or no scaring.

### EXAMPLE XIII

Another exemplary embodiment of the invention includes the use of the composition as an aid in the treatment of athletes feet. The composition was applied twice daily to clean dry feet of individuals suffering from discomfort and odor associated with the infection. Individuals reported that the composition minimized the discomfort and/or soreness and that the odor was reduced or removed.

### EXAMPLE XIV

Another exemplary embodiment of the invention includes the use of the composition as a lip gloss and/or treatment or preventative agent for chapped lips. The composition was applied to chapped, sunburned and/or wind burned lips and was reported to relieve the symptoms. In addition, the composition may be applied to an individual's lips, either alone or with lipstick or tint, to produce a desirable gloss and keep the lips soft.

All references, including publications, patents, and patent applications, cited herein are hereby incorporated by reference to the same extent as if each reference were individually and specifically indicated to be incorporated by reference and were set forth in its entirety herein.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the meaning of the appended claims.

What is claimed is:

1. A topical composition comprising:
a mixture of dimethicone and cyclomethicone;
mineral oil;
Aloe vera gel;
a vitamin E source, wherein the vitamin E source is an oil;
a vitamin A source, wherein the vitamin A source is an oil; and
water in an amount from about 0.05% to about 0.5% of the volume of the dimeticone and cyclomethicone mixture.

2. The topical composition of claim 1, wherein the mixture of dimethicone and cyclomethicone is from about 60:40 to about 70:30 (vol.:vol.) dimethicone:cyclomethicone.

3. The topical composition of claim 1, wherein the mineral oil is in an amount from about 0.2% to about 4% of the volume of the dimeticone and cyclomethicone mixture.

4. The topical composition of claim 1, wherein the Aloe vera gel is in an amount from about 0.2% to about 4% of the volume of the dimeticone and cyclomethicone mixture.

5. The topical composition of claim 1, wherein the vitamin E source is in an amount from about 0.2% to about 4% of the volume of the dimeticone and cyclomethicone mixture.

6. The topical composition of claim 1, wherein the vitamin A source is in an amount from about 0.1% to about 2% of the volume of the dimeticone and cyclomethicone mixture.

7. The topical composition of claim 1, wherein the mineral oil, Aloe vera gel, and vitamin E source are in equal amounts.

8. The topical composition of claim 1, further comprising a scented oil an amount of from about 0.1% to about 2% of the volume of the dimeticone and cyclomethicone mixture.

9. The topical composition of claim 1, wherein:
the mineral oil, Aloe Vera gel, and vitamin E source are each in an amount from about 0.2% to about 4% of the volume of the dimeticone and cyclomethicone mixture; and
the vitamin A source is in an amount from about 0.1% to about 2% of the volume of the dimeticone and cyclomethicone mixture.

10. The topical composition of claim 9, wherein the mineral oil, Aloe Vera gel, and vitamin E source are each in an amount of about 3% to about 4% of the volume of the dimeticone and cyclomethicone mixture.

11. The topical composition of claim 10, wherein the vitamin A source is in an amount of about 1% to about 2% of the volume of the dimeticone and cyclomethicone mixture.

12. The topical composition of claim 11, wherein the water is in an amount of about 0.2% to about 0.3% of the volume of the dimeticone and cyclomethicone mixture.

13. The topical composition of claim 9, wherein the mineral oil, Aloe Vera gel, and vitamin E source are each in an amount of about 1% to about 2% of the volume of the dimeticone and cyclomethicone mixture.

14. The topical composition of claim 13, wherein the vitamin A source is in an amount of about 1% to about 2% of the volume of the dimeticone and cyclomethicone mixture.

15. The topical composition of claim 14, wherein the water is in an amount of about 0.2% to about 0.3% of the volume of the dimeticone and cyclomethicone mixture.

16. The topical composition of claim 15, further comprising a scented oil in an amount of about 0.1% to about 1.4% of the volume of the dimeticone and cyclomethicone mixture.

17. The topical composition of claim 16, wherein the scented oil is an essential oil derived from lavender.

18. The topical composition of claim 1, wherein:
the mineral oil, Aloe Vera gel, and vitamin E source are each in an amount from about 0.3% to about 3% of the volume of the dimeticone and cyclomethicone mixture; and
the vitamin A source is in an amount from about 0.3% to about 1.6% of the volume of the dimeticone and cyclomethicone mixture.

19. A method of maintaining or improving a skin or hair condition, the method comprising:
applying a topical composition of claim 1 to the skin or hair of a subject, thereby maintaining or improving the skin condition of the subject.

20. The method according to claim 19, comprising applying a topical composition of claim 9.

21. The method according to claim 19, comprising applying a topical composition of claim 13.

22. The method according to claim 19, comprising maintaining or improving a skin or hair condition selected from the group consisting of burns, sunburns, eczema, psoriasis, dry or bleeding cuticles, dermatitis, athletes foot, razor rash or razor burn, cold sores, dry or chapped lips, dry or frayed hair, and combinations thereof.

23. A method of treating pain associated with a subject's skin or joints, the method comprising:
applying a topical composition of claim 17 to the skin or hair of a subject; and
treating pain associated with the skin or joints of the subject.
